(19) United States
(12) Patent Application Publication
(10) Pub. No.: US 2007/0160223 A1
(43) Pub. Date: Jul. 12, 2007

(54) Method and apparatus for noise canceling headphones

(76) Inventor: Francis John Cusack JR., Groton, MA (US)

Correspondence Address: FRANCIS CUSACK, 174 DUCK POND DRIVE, GROTON, MA, 01450, US

(21) Appl. No.: 11/323,928

(22) Filed: Jan. 3, 2006

(51) Int. Cl. A61F 11/06

(52) U.S. Cl. 381/71.1

(57) ABSTRACT

An invention is described for a personal noise canceling system that consists of noise canceling headphones, a noise cancellation electronics module and a personal computing device. One embodiment of the invention places the noise canceling electronics module in a USB connector fob, which serves as a personal computing device, and also acts as the connection interface between the headphones and a complementary computing device. Another embodiment places the noise canceling electronics module within a decoupled personal computing device where built in audio electronics, software, firmware and power source may be leveraged for implementation of signal conditioning and processing in support of noise cancellation. It is envisioned that desirable audio signals may be injected into the noise cancellation system from a variety of sources, including but not limited to, the USB connector fob (such as digital music audio), a complementary computing device (such as DVD sound track) or an external audio source (such as airline audio channel).

### CROSS REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF INVENTION

Noise canceling headphones have proven to be a popular device for travelers, office workers and others who wish a reprieve from the distraction of unwanted background noise or who wish to hear with enhanced clarity the details of a particular audio source while in the presence of background noise that would otherwise detract from or mask the desired audio.

Traditional noise canceling headphones use a combination of active and passive noise reduction techniques. Passive noise cancellation techniques are used first to attenuate the noise that reaches the ear, and active noise cancellation techniques are then used to introduce noise of a type that will cancel the ambient noise and thereby minimize the net sound that ultimately reaches the ear.

Passive noise reduction is achieved by the shape and sound absorbing characteristics of the earpiece cups. Large cups that cover the ear completely are more effective, and more cumbersome, than smaller ear cups. Cups filled with sound absorbing material, such as gel, aids in reducing the sound levels that reach the ear and in turn relax the requirements on the active noise canceling electronics and transducers.

Active noise cancellation can be achieved in a number of ways and to degrees of effectiveness. But most approaches have basic similarities. A microphone within close proximity to the ear, such as within a headphone ear cup, detects audio signals within the audible spectrum (signal amplitudes and frequencies that can be detected by the human ear). This detected audio signal close to the ear, or the error signal, is used in a feedback loop. Electronics condition the error signal and produce a copy that has the same amplitude at each tone (i.e. same spectrum), but shifts the phase of the error signal by 180 degrees. This equal (in amplitude) and opposite (in phase) correction signal is then fed back to the listening environment via the speaker transducer in the headphone. When the correction signal is passed to the speaker the analog electrical signal is converted to mechanical motion which produces sound that is exactly equal and opposite to the sound detected at nearly the same instant by the built in microphone. The result of propagating the correction sound in the presence of the ambient detected noise is that the two sounds interfere and cancel, and the noise level detected by the listener is noticeably reduced from that which would heard in the absence of active noise cancellation. The servo loop bandwidth, temporal response, frequency response and other characteristic parameters are chosen to optimize the active noise canceling effect taking into account the technical performance of the passive noise cancellation sub system, and the anticipated noise characteristics to be encountered.

A drawback to the current noise canceling headphones is that they are typically stand alone products that house their own power supply, noise cancellation electronics, microphones and headphones. As a result the units are expensive and cumbersome.

An improvement to the current state of the art could be realized by an invention that takes advantage of some of the intrinsic features of a personal computer. For example desktop computers and laptops have power supplies that produce DC voltages that could be used to power the noise canceling headphones and thereby eliminate need for a battery pack.

Personal computers also often have integral microphones that may be used to enhance the noise canceling headphones. And naturally personal computers have processing, analog to digital conversion, and digital to analog conversion capabilities that may be used to enhance if not completely implement the noise canceling signal conditioning and correction signal generation. Finally, the USB connector is an elegant connection interface to pass power and signals between the personal computer and the headphone assembly that may or may not be employed. If it is employed, interesting extensions of the invention are envisioned, such as the inclusion of a digital music player, or simply digital storage on the USB connector fob.

### BRIEF SUMMARY OF THE INVENTION

An invention is described for a personal noise canceling system that consists of noise canceling headphones, a noise cancellation electronics module and a personal computing device. One embodiment of the invention places the noise canceling electronics module in a USB connector fob, which serves as a personal computing device, and also acts as the connection interface between the headphones and a complementary computing device. Another embodiment places the noise canceling electronics module within a decoupled personal computing device where built in audio electronics, software, firmware and power source may be leveraged for implementation of signal conditioning and processing in support of noise cancellation. It is envisioned that desirable audio signals may be injected into the noise cancellation system from a variety of sources, including but not limited to, the USB connector fob (such as digital music audio), a complementary computing device (such as DVD sound track) or an external audio source (such as airline audio channel).

### BRIEF DESCRIPTION OF THE SEVERAL VIEWS OF THE DRAWING

FIG. 1: Noise Canceling Module (NCM) is integral to Personal Computing Device (PCD). Connection between Noise Canceling Headphones (NCH) and PCD is implemented with a USB connector fob.

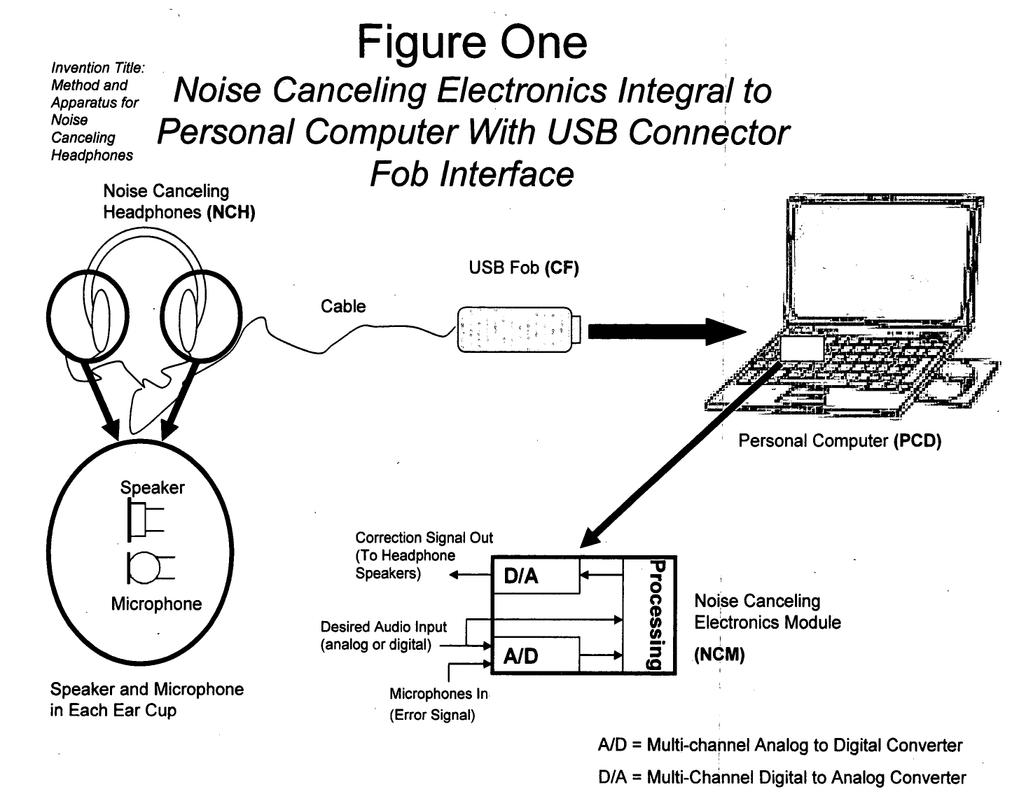

FIG. 2: Noise Canceling Module (NCM) is integral to Personal Computing Device (PCD). Connection between Noise Canceling Headphones (NCH) and PCD is implemented with traditional audio connectors.

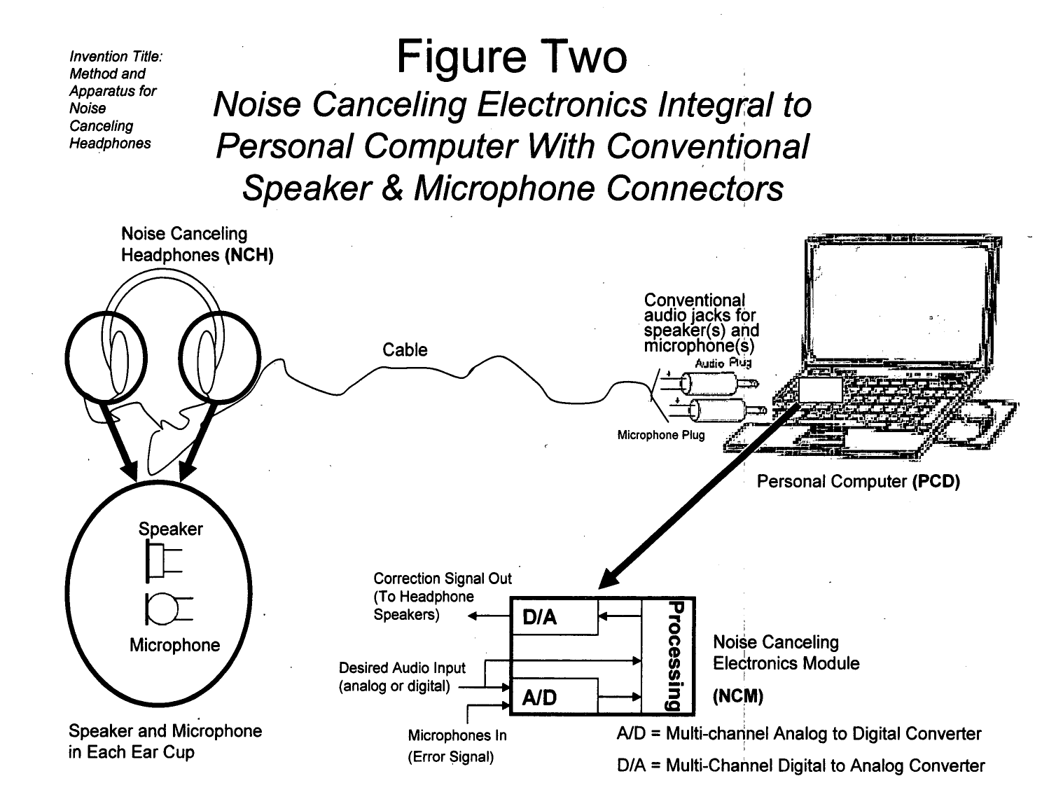

FIG. 3: Noise Canceling Module (NCM) is integral to Connector Fob (CF), in this example a USB type connector fob is used. Connection between Noise Canceling Headphones (NCH) and PCD is implemented with same USB connector fob.

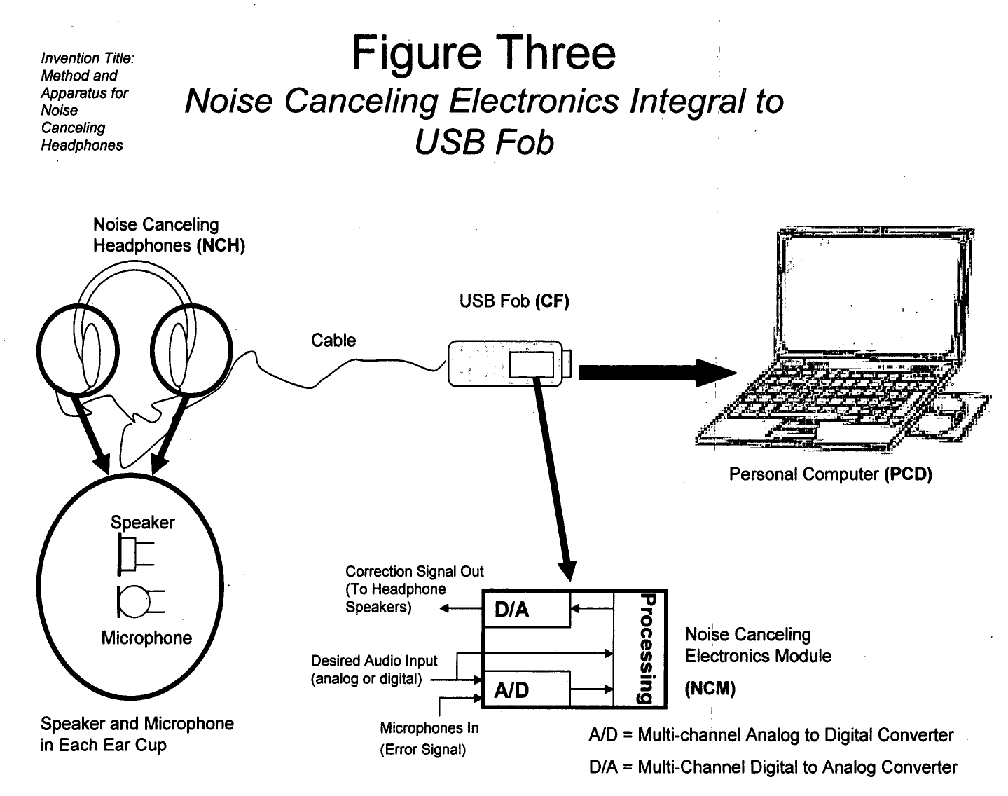

FIG. 4: Noise Canceling Module (NCM) is integral to Connector Fob (CF), in this example a USB type connector fob is used. Also resident on CF is a digital music player. Connection between Noise Canceling Headphones (NCH) and PCD is implemented with same USB connector fob.

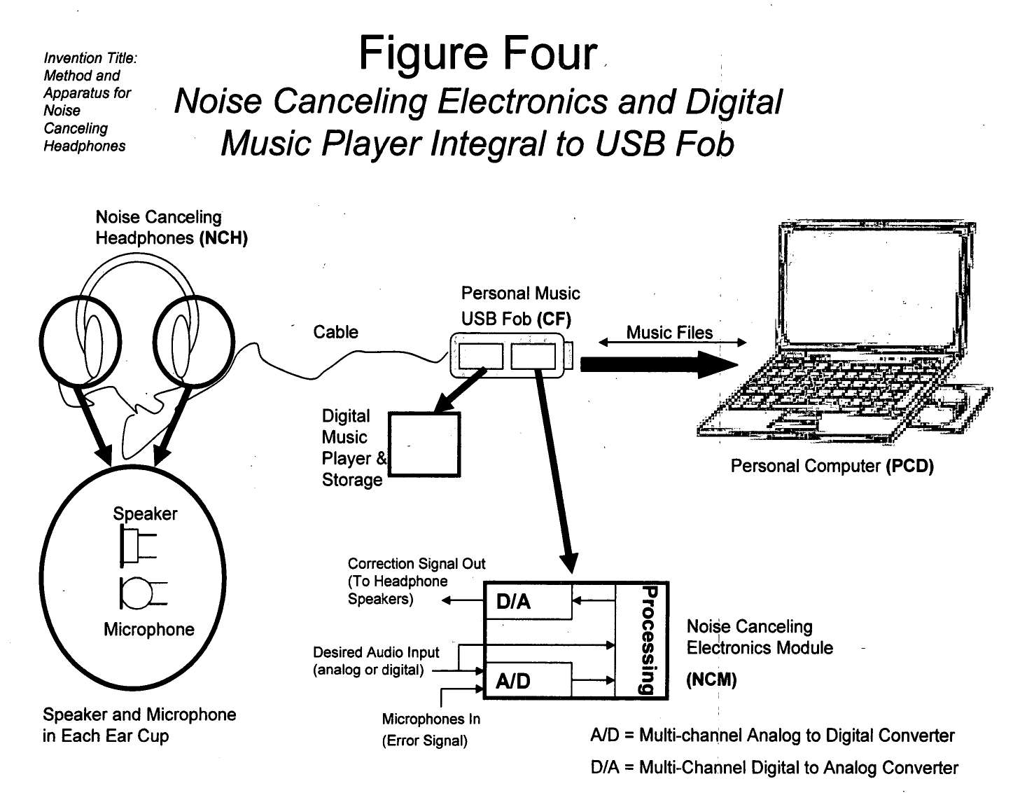

FIG. 5: Noise Canceling Module (NCM) is integral to PCD in this example. A USB type connector fob is used to house a digital music player. Connection between Noise Canceling Headphones (NCH) and PCD is implemented with same USB connector fob.

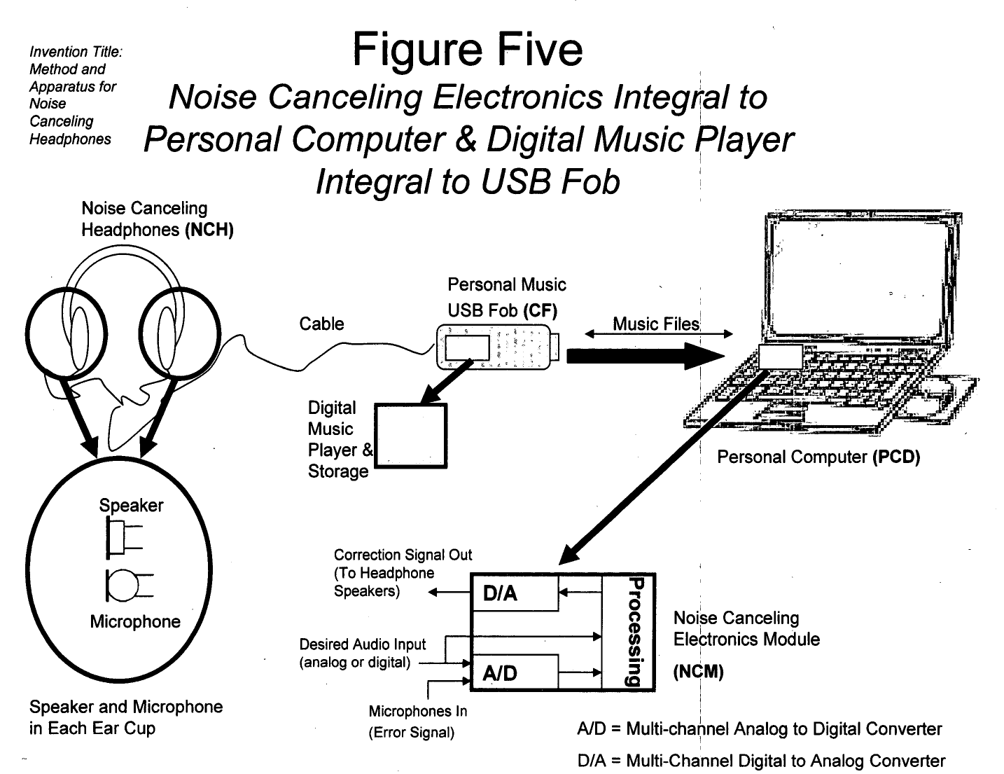

### DETAILED DESCRIPTION OF THE INVENTION AND PREFERRED EMBODIMENTS

There are many embodiments that can be envisioned to effectively implement the invention. Several are described herein in detail, but it will be appreciated by those skilled in the art that there are many variations on the fundamental configuration that are within the spirit and scope of this invention.

Noise Cancellation Module Integral to Personal Computing Device; Headphone to Personal Computing Device via USB Connector Fob

The preferred embodiment, shown in Figure One, consists of a Personal Computing Device, referred to herein as the PCD (e.g. desktop, laptop, mobile phone) and a Noise Canceling Headphone assembly, referred to herein as the NCH, that connects to the PCD. The connection between the PCD and the NCH is a cable terminated either directly within the PCD, or via a Connector Fob referred to herein as CF, such as a USB connector fob for example. All software, audio electronics, computing electronics, signal conditioning electronics and other devices, electronics and software necessary to implement to function of noise cancellation are herein described as components of the Noise Cancellation Module, or NCM (The NCM is a functional description, and may physically exist in one device or among devices).

A small microphone resides in each of the NCH ear cups. Noise signals are detected by the microphones integral to the ear cup and passed to the CF. Here the noise reduction module resident within the CF conditions the signal to be equal in amplitude and opposite in phase when played back through the headphone speakers. The corrected signal is passed to the NCH, where the signal is converted to sound by the speaker transducers and mixed with the resident ambient noise. The ambient noise is cancelled by the correction signal converted to sound by the NCH speakers. After cancellation, the residual noise is detected and again passed back to the cancellation electronics and the process repeats in a closed loop servo fashion to sustain maximum noise suppression in real time and in the presence of changing background noise amplitude and frequencies.

In this embodiment, the NCM resides on the PCD to reduce the requirements on noise canceling electronics external to the PCD. In the preferred configuration, the error signal is detected by microphones in the ear cup and passed through the USB CF to the PCD's internal audio electronics. Here the signal is either processed with analog electronics, or is converted to a digital format via the PCD's analog to digital converter and processed digitally. After processing, the analog correction signal (in the case of a digital domain process the signal would be converted to analog via the PCD's digital to analog converter) is passed back to the headphones to drive the headphone speakers and cancel the ambient noise, thereby achieve a reduction in unwanted background noise.

Desired audible signals may be superimposed and enjoyed in the relative absence of background noise. Desirable audio signals may originate from the PCD. Examples are; traditional software application sounds (e.g. beeps, chimes, etc.), Voice over IP (VOIP) phone conversations, or may be entertaining in nature such as music from a CD, stored digital formats such as .WAV or MP3, or may be the soundtrack to a DVD movie and so on. Alternatively, the audio source may be external to the PCD, such as an MP3 player, a cell phone, an external CD player or an airline audio source.

Noise Cancellation Module Integral to Personal Computing Device; Headphone to Personal Computing Device via Traditional Audio Connectors.

In another embodiment, shown in Figure Two, the NCH connects to the PCD via traditional audio jacks instead of a USB type connection.

Noise Cancellation Module Integral to USB Connector Fob

In another embodiment, shown in Figure Three, the NCM and associated noise canceling electronics and software (or firmware) reside within the CF. Power is supplied from the PCD to the noise canceling electronics within the CF.

Noise Cancellation Module and Digital Music Player Integral to USB Connector Fob

In another embodiment, shown in Figure Four, the NCM and a digital music player reside within the CF. Power is supplied from the PCD to the noise canceling electronics within the CF. In this configuration, digital music may be stored and retrieved from the USB CF, and desired audio content may be superimposed on the cancellation signals to provide an enhancement listening experience. Whether the CF is connected or disconnected from the PCD, operation of either noise cancellation or music generation, or a combination of the two may be employed. While disconnected from the PCD, battery power enables continued operation.

Noise Cancellation Module Integral to PCD and Digital Music Player Integral to USB Connector Fob

In another embodiment, shown in Figure Five, the NCM resides on the PCD and a digital music player reside within the CF. Power is supplied from the PCD to the digital music player within the CF. In this configuration, digital music may be stored and retrieved from the USB CF, and desired audio content may be superimposed on the cancellation signals to provide an enhancement listening experience. When the CF is disconnected from the PCD, operation of music generation is enabled by CF battery power.

Those skilled in the art will recognize that there are many more combinations of the NCH, NCM, PCD and CF that can be employed.

1. The invention provides a means of actively suppressing or canceling unwanted background or ambient noise within proximity to the users ear or ears through the use of Noise Canceling Headphones (NCH), and a Noise Canceling Module (NCM) that resides either wholly or partially on a Personal Computing Device (PCD).
Noise Canceling Headphones (NCH)

2. The NCH in claim one may employ a cable to provide ease of movement and flexibility while the NCH are worn.

3. The NCH in claim one may provide a means of passively suppressing unwanted background noise from reaching the ear or ears by careful design of a headphone assembly that gives consideration to the shape and material composition of the ear cups to absorb, reflect or in any way attenuate the level of unwanted background noise that reaches the ear or ears. The NCH ear cups may be of the type that completely cover the ear, partially covers the ear, or insert into the ear. The NCH in claim one may consist of one ear cup or two ear cups (one for each ear), and contains a speaker transducer in each ear cup.

4. The NCH in claim one may contain an integral microphone in the ear cup or in each ear cup to support noise cancellation.

5. The NCH in claim one may contain a boom style or other microphone external to the internal or acoustically shielded region of the ear cup or ear cups to support noise cancellation or collection of desirable audio signals.
Noise Canceling Module (NCM)

6. The Noise Canceling Module in claim one takes in and conditions background noise signals and produces the correction signal to be fed to the NCH to achieve a reduction in unwanted noise. The NCM comprises all software, firmware, hardware, electronics, and any and other devices and logic necessary for the processing of active noise cancellation. This module is responsible for taking in signals of unwanted background noise and producing a correction signal of amplitude, phase and frequency that will, when played through the NCH speakers, produce a reduction in unwanted noise as received by the ear(s).

7. The NCM in claim one may receive and/or process either analog signals, digital signals, or a combination of the two.

8. The NCM in claim one may employ analog electronics, digital electronics, software, firmware, logic and any or all other techniques understood be employed in the implementation of active noise cancellation.

9. The NCM in claim one may physically reside all in one location, such as a personal computing device or a connector fob (for example a USB connector fob that provides a means of connecting the NCH and the PCD), or may be decoupled into two or more components that may physically reside in different locations, such as a combination of a personal computing device and a connector fob.

10. The NCM in claim one may receive error signals from a microphone or microphones in a variety of locations. Error signal sources may include, but are not limited to, NCH microphones, either within the ear cup(s), on the ear cup(s) and/or external to the ear cup(s), microphones connected to the PCD or built into the PCD, and/or connector fob microphones.

11. The NCM in claim one may receive desirable audio signals that should not be cancelled, but may be conditioned or processed within the NCM, or external to the NCM, prior to forwarding to the NCH. Desirable audio signals may include, but are not limited to, those from microphone(s), such as a boom microphone mounted on the NCH, audio signals from a personal computing device such as a mobile phone, personal music player (e.g. MP3), audio signals generated by the connector fob, and/or audio signals generated by the PCD. Audio signals may be of analog or digital origin.
Personal Computing Device (PCD)

12. The PCD in claim one is any device that supports either all or part of the functionality of the NCM of claim one. Examples are, but are not limited to; personal desktop computer, laptop computer, handheld personal computing device, cell phone, personal digital music player, portable storage device (such as a USB memory stick) or a connector fob (such as that described to connect the NCH and the PCD).

13. The PCD in claim one may be equipped with a microphone or microphones that may be employed in support of noise cancellation.

14. The PCD in claim one may be equipped with software, firmware, hardware, analog and/or digital electronics and any or other means that may be employed in support audio signal conditioning and processing and noise cancellation.

15. The PCD in claim one may be equipped with a power source that may be employed in support of noise cancellation.
Connector Fob (CF)

16. The NCH cable in claim two may terminate in a Connector Fob (CF). The CF may either serve as the PCD of claim one, or serve as an interface between the NCH of claim one and the PCD of claim one.

17. The CF in claim sixteen may possess the means to receive, store and/or provide digital and analog data and/or signals from external devices. An example of an interface between the CF and a PCD of claim one is, but is not limited to, a USB connection. An example of the CF receiving analog data may be via a microphone to detect either desirable or undesirable audio signals for forwarding to the NCM of claim one.

18. The NCM of claim one may either completely reside, partially reside, or not reside on the CF.

19. The CF in claim sixteen may possess a digital music player (such as MP3) that produces desirable audio signals to the NCM of claim one. These signals may be passed to the NCM of claim one either with an external connection or internally within the CF with a direct connection.

20. The CF in claim sixteen may possess a power source, such as a conventional battery or a rechargeable battery, or may draw power from an external power source, such as a complementary PCD.
